(19) United States
(12) Patent Application Publication
(10) Pub. No.: US 2015/0322394 A1
(43) Pub. Date: Nov. 12, 2015

(54) SYSTEM AND METHOD OF PRODUCING GLUCOMYLASES AND/OR PROTEASES

(71) Applicant: City University of Hong Kong, Kowloon (HK)

(72) Inventors: Sze Ki Carol Lin, Hong Kong (HK); Zhi Quan Lin, Dongguan City (CN)

(73) Assignee: City University of Hong Kong, Kowloon (HK)

(21) Appl. No.: 14/270,402

(22) Filed: May 6, 2014

(51) Int. Cl. C12M 1/00; C12M 1/02; C12N 9/62; C12M 1/12; C12N 9/34; C12M 1/16; C12M 1/34

(52) U.S. Cl. CPC C12M 21/14; C12M 21/16; C12M 29/06; C12M 41/18; C12M 41/40; C12M 23/06; C12N 9/2428; C12N 9/62

(57) ABSTRACT

The present invention is concerned with a system of production of glucoamylases and/or proteases. The system has a packed bed solid state fermentation bioreactor. The bioreactor is adapted to contain and to operate using organic waste as fermentation substrate. The bioreactor is provided with spray nozzle for controlling moisture content of the fermentation substrate, means for supplying air to pass through packed bed of the fermentation substrate in said bioreactor, means for analyzing gas content in said bioreactor, means for monitoring temperature in said bioreactor, means for controlling temperature of said bioreactor, a port via which a sample in said bioreactor is obtainable, and means for harvesting enzymatic solution from said bioreactor, the enzymatic solution containing the glucoamylases and/or proteases.

### FIELD OF THE INVENTION

The present invention is concerned with a system and a method for producing glucoamylases and/or proteases by fermentation of organic wastes.

### BACKGROUND OF THE INVENTION

Organic waste and in particular food waste has been a prevalent problem among many developed countries such as the US, the UK, Japan and Korea. The problem is also very serious in many Asian cities. The situation is particularly problematic in densely populated cities like Hong Kong because limited sites which can be used for landfills.

In the example of Hong Kong, there is about 3,584 tons of food waste generated every day. One third of this food waste is originated from commercial and industrial sector, and the remaining comes from domestics dwelling. Statistics show that food waste accounts for around 40% to the total municipal solid waste generated in Hong Kong.

Organic wastes in general and food wastes in particular pose major challenges to environmental management for many reasons. First, there is depleting supply of suitable landfill. Second, incineration of food wastes is unsuitable due to high water content and the possibility of dioxin generation. Dioxin is carcinogenic and teratogenic in certain animals. Third, treatment of food wastes by conventional treatment methods often would cause other types of pollution.

Recycling could be one way of relieving burden on landfills by food wastes. However, conventional recycling method for food wastes has been employed for the production of animal feed and fertilizer. Unfortunately, large amounts of wastewater are often generated when desalting the food wastes for fertilizer production and animal feeds, and that would cause further hygiene problems.

The present invention seeks to address the aforementioned problems by turning organic wastes and particular food wastes into useful products without causing un-manageable side effects, or at least to provide a useful alternative to the public.

### SUMMARY OF THE INVENTION

According to a first aspect of the present invention, there is provided a system of production of glucoamylases and/or proteases, comprising a packed bed solid state fermentation bioreactor, wherein the bioreactor is adapted to contain and to operate using organic waste as fermentation substrate, and is provided with spray nozzle for controlling moisture content of the fermentation substrate, means for supplying air to pass through packed bed of the fermentation substrate in the bioreactor, means for analyzing gas content in the bioreactor, means for monitoring temperature in bioreactor, means for controlling temperature of the bioreactor, a port via which a sample in the bioreactor is obtainable, and means for harvesting enzymatic solution from the bioreactor, the enzymatic solution containing the glucoamylases and/or proteases.

Preferably, the bioreactor may be adapted to receive and operate using at least one of Aspergillus awamori and Aspergillus oryzae as fermentation agent.

The fermentation substrate of the system may be bakery waste. The bakery waste may include at least one of bread waste, cake waste and pastry waste.

In an embodiment, the spray nozzle may be adapted to introduce sterilized water in said bioreactor. The spray nozzle may be arranged at top region of said bioreactor. The spray nozzle may be adapted to maintain moisture content of the fermentation substrate in the bioreactor at 60-80 wt %.

In one embodiment, the air supply means may include filtering means to prevent contamination. The air supply means may be adapted to introduce filtered air from bottom region of the bioreactor.

The system may comprise means for receiving air exiting the bioreactor. The air receiving means may be connected to, and upstream of the air analyzing means. The system may comprise an outlet at a top region of the bioreactor via which air exits to the air receiving means.

The air supply means may be adapted to moist air before introducing air in the bioreactor.

Suitably, the temperature control means may be in the form of a water jacket for removing heat generated during operation of the system.

Advantageously, the bioreactor may be provided with a port via which the enzymatic solution exits. The port may be arranged at a bottom region of said bioreactor.

The proteases produced by the system may include at least one of serine proteases, cysteine proteases, aspartic proteases and metalloproteases.

Preferably, the fermentation reactor may be of cylindrical profile defining a cavity to contain the fermentation substrate.

In a preferred embodiment, the fermentation substrate may be provided with a plurality of platform members laterally extending from side wall of the fermentation reactor and spaced apart from each other, the platform members for supporting the fermentation substrate.

According to a second aspect of the present invention, there is provided a method of producing glucoamylases and/or proteases, comprising steps of providing a packed bed solid state fermentation bioreactor defining a cylindrical body, providing organic waste as fermentation substrate for use in the bioreactor, providing platform members in the bioreactor and laterally extending from side wall of the bioreactor, the platform members supporting the fermentation substrate and spaced apart from each other, introducing Aspergillus awamori and Aspergillus oryzae in the bioreactor for producing enzymes acting as biocatalysts for solid state fermentation using the organic waste, and providing a spray nozzle for controlling moisture content of the fermentation substrate.

### BRIEF DESCRIPTION OF THE DRAWINGS

Some embodiments of the present invention will be explained below, with reference to the accompanied drawings, in which:—

FIG. 1 is a photographic image of an embodiment of a fermentation bioreactor according to the present invention;

FIG. 2 illustrates interior structure of another embodiment of a fermentation bioreactor according to the present invention;

FIG. 3 is a schematic diagram showing different components of the fermentation bioreactor of FIG. 2;

FIG. 4 is a graph showing the relationship of glucoamylase activity (from sample of fermentation substrate) of A. awamori and fermentation time in an experiment;

FIG. 5 is a graph showing the relationship of protease activity (from sample of fermentation substrate) of A. oryzae and fermentation time in an experiment;

FIG. 6 is a graph showing the relationship of glucoamylase activity (from sample of fermentation substrate) and reaction pH in an experiment;

FIG. 7 is a graph showing the relationship of glucoamylase activity (from sample of fermentation substrate) and reaction temperature in an experiment;

FIG. 8 is a graph showing the relationship between thermo-stability of glucoamylase and time during fermentation in an experiment;

FIG. 9 is a graph showing the relationship of glucose concentration and digestion time during fermentation in an experiment;

FIG. 10 is a graph showing the relationship of Free Amino Nitrogen (FAN) and digestion time during fermentation in an experiment;

FIG. 11 is a graph showing the relationship of glucose concentration as a result enzymatic hydrolysis in a mixed enzyme solution environment in an experiment;

FIG. 12 is a graph showing the relationship of glucose yield and digestion time in fermentation of food waste in an experiment;

FIG. 13 is a graph showing the relationship of glucose yield and digestion time in fermentation of food waste in experiments using only A. awamori, and A. awamori and A. oryzae, respectively; and

FIG. 14 is a graph showing the relationship of glucomylase activity and time in fermentation experiments with air flow of 1.6 vvm and 1.0 vvm, respectively.

### DETAILED DESCRIPTION OF THE INVENTION AND EMBODIMENTS THEREOF

The present invention makes use of fermentation in fermentation bioreactor for converting organic wastes and in particular food wastes into valuable protease and/or glucoamylase.

While different organic wastes may be used, studies leading to the present invention show that food wastes are generally useful in producing protease and/or glucoamylase. Among different food wastes, in specific embodiments, bakery wastes are found to be particularly suitable as substrate for use in packed bed solid state fermentation of the present invention. During the course of the research, it is found that bakery wastes are high in carbohydrates and have an appropriate content of other nutrients, and these can support the efficient microbial growth during fermentation, and thus producing protease and/or glucoamylase. It is found that, during fermentation and hydrolysis of food wastes, enzymes of glucoamylase and protease are secreted for substrate digestion to support the fungal growth. These enzymes are useful because glucoamylase and protease are among the most widely used industrial enzymes which can be applied in industries like leather, brewing, textile, paper, distilling, food processing, pharmaceuticals, waste decomposition, detergents, etc. The enzymes produced are utilized in production of nutrients rich hydrolysates which are then fermented with microorganisms to make chemicals like bio-ethanol, succinic acid, polyhydroxybutyrate (PHB), etc. Besides reducing food waste by solid state fermentation, different kinds of value-added products are produced which can reduce dependency on petroleum by-products.

Bakery wastes including cake, bread and pastry can all be used as substrate for the solid state fermentation. In preferred embodiments of the present invention, the microorganisms chosen are two fungi: Aspergillus awamori and Aspergillus oryzae.

Preliminary experiments were performed on petri dishes first using the three substrates separately for the production of proteases and glucoamylases. The best substrate that gave the highest enzymatic activity was then selected for a scale up fermentation process for the production of multi-enzymes solution in a solid state fermentation bioreactor. Enzyme extraction was performed for every day sampling. The glucoamylase and proteases activities were determined by glucoamylase and protease assays respectively. Glucoamylase and protease assays were analyzed using Analox GL6 Glucose Analyzer and ninhydrin colorimetric method respectively.

It is to be noted that the present invention is concerned with the use of solid state fermentation (SSF) and not submerged fermentation (SMF). Studies leading to the present invention have found that the SMF would lead to suboptimal growth conditions for certain microorganisms such as fungi Aspergillus. Further, SMF would produce large amount of effluents in downstream processes. On the other hand, SSF has been identified because of its lower production cost and it can foster more stable fungal products. Further, SSF involves the growth of microorganisms on moist solid particles or fermentation substrate in the absence or nearly in the absence of free flowing water.

The following table summarizes the biotechnological advantages of packed bed solid state fermentation for use in the present invention.

TABLE 1
Biotechnological advantages of solid state fermentation

| Advantages | Effect |
|---|---|
| Lower moisture requirement | Suitable for fungal growth |
| | Reduction in effluent volume |
| | Reduction in contamination risk |
| Higher yields and concentration of the | Lower downstream costs |
| end product | |
| Easier recovery of products | |
| Simulation of the natural environment | Better performance of cultivated |
| | microorganisms |
| High-volume productivity | Smaller fermenter volumes |
| Simple set-up | Lower cost |
| Low energy demand for heating/ | |
| cooling | |
| Easy aeration | |
| Utilization of cheap and widely | |
| available agricultural residues or food | |
| waste as substrate | |
| Little/no pre-treatment of the waste | |
| substrate before fermentation | |

SSF is advantageous when compared with other types of fermentation in the context of enzyme productions of the present invention, in that the production cost is lower and the production yield is higher. The enzymes are produced concomitantly in single stage fermentation and they can be applied in production of detergent, bio-ethanol, animal feed, etc.

While bacteria, yeast and fungi can all be used in SSF, the present invention has adopted fungi as fermentation agent or more accurately biocatalyst. Among thousands of the species in the kingdom of fungi, Aspergillus awamori and Aspergillus oryzae have been selected because studies leading to the present in invention have shown that they can produce glucoamylases and proteases in relatively high concentrations.

While different agricultural waste such as grape pomace or a wide variety of natural plant crops such as wheat, corn, rice, cereals, wheat flour, etc. may be used as fermentation substrates of the present invention. The present invention has made use of food wastes, and in particular bakery wastes such as bread wastes, pastry wastes and cake wastes. These wastes are easily collectable from restaurants, hotels, eateries and bakeries in developed countries. Bakery wastes contain the nutritious contents required for fungal growth during in SSF. Bakery waste is mainly divided into three categories: bread, cake and pastry. The use of SSF in the present invention allows the production of multi-enzyme solutions.

The enzymes produced from solid state fermentation, glucoamylase and protease, are also used for enzymatic hydrolysis of food wastes. Glucoamylase, also the name of amyloglucosidase, is the major enzyme produced by strains of the fungal genus Aspergillus and also a major industrial enzyme used in the saccharification step of starch hydrolysis. Thus, Aspergillus awamori has been chosen as a preferred microorganism in the present invention. Glucoamylase has a strong affinity for starch and to hydrolyse them to glucose effectively. Studies have shown that the optimal reaction temperature and pH for the enzymatic hydrolysis are 55° C. and 4.5-5.0, respectively.

Proteases are major industrial enzymes that are produced in large quantities by strains of the fungal genus Aspergillus. Aspergillus oryzae is another preferred organism in the present invention. According to the catalytic mechanisms of protease, they are generally divided into four groups: serine proteases, cysteine proteases, aspartic proteases and metalloproteases. In clinical and biochemistry laboratories each type of proteases may be applied to carry out specific and highly selective modifications of proteins through limited hydrolysis, but basically they are regarded as degradative enzyme. Studies have shown that the optimal reaction temperature for the enzymatic hydrolysis is 55° C., and pH of the solvent had very little effect on protease activity.

During the enzymatic hydrolysis, food wastes are digested by the enzymes, and finally break down the macromolecules into monomers. Specifically, starch is broken down to glucose by glucoamylases and protein is broken down into amino acids by proteases.

The following table illustrates the nutrient compositions of typical bakery wastes.

TABLE 2
The nutrient compositions of typical bakery items

| Bakery | Carbohydrates | Proteins | Lipids |
|---|---|---|---|
| Waste | (mg/g) | (mg/g) | (mg/g) |
| Bread | 468 | 90 | 89 |
| Cake | 620 | 190 | 170 |
| Pastry | 335 | 352 | 71 |

The substances, glucoamylases and proteases, produced by a system in accordance with the present invention are valuable because they are among the most widely used industrial enzymes which can be applied in industries like leather, brewing, textile, paper, distilling, food processing, pharmaceuticals, waste decomposition, detergents, etc. The enzymes produced are utilized in production of nutrient-rich hydrolysates which are then fermented with microorganisms to make chemicals like bio-ethanol, succinic acid, polyhydroxybutyrate (PHB), etc.

In preferred embodiments of the present invention, two filamentous fungi are selected for the fermentation, Aspergillus awamori and Aspergillus oryzae. Studies leading to the present invention show that filamentous fungus can produce a wide range of extracellular enzymes at low moisture contents efficiently and the low water environment in SSF suits fungal growth well. Moreover, filamentous fungi can grow on complex solid substrates. Their hyphal mode of growth allows penetration into the solid material and production of a wide variety of extracellular enzymes which are produced to break down macromolecules. Filamentous fungi can grow simultaneously at the surface and within the solid substrate which makes them ideal for SSF. Since filamentous fungi are more suitable for the habitat in SSF and possess more advantages than other microorganisms, it is a popular choice to be the host in SSF.

Preparation of the Fungus Aspergillus oryzae
In an experiment to demonstrate the efficacy of the present invention, a strain of A. oryzae was isolated from a soy source starter supplied by the Amoy Food Limited in Hong Kong. A stock solution of A. oryzae (6.31×10⁶ M) was stored at −80° C. refrigerator for preservation.
Aspergillus awamori
A. awamori is classified by the Common Wealth Mycological Institute as Aspergillus niger complex. The A. awamori used in experiments leading to the present invention was obtained from the American Type Culture Collection (ATCC). A stock solution of the A. awamori (2.85×10⁷ M) was stored at −80° C. refrigerator for preservation.

Characteristics of bioreactors used for solid state fermentation are explained in light of other types of bioreactors.

TABLE 3
Basic design feature of Solid State Fermentation

| | No mixing | Continuous mixing, or |
|---|---|---|
| | (or very | frequent |
| | infrequent) | intermittent mixing |
| No forced aeration (air | Tray chamber | Rotating drum, Stirred |
| passes around bed) | | drum |
| Forced aeration (air | Packed bed | Gas-solid fluidized bed, |
| blown forcefully through | | Stirred bed, Rocking drum |
| the bed) | | |

Packed-bed solid state fermentation bioreactor operates under static conditions with forced aeration have been identified to suit particular production of the enzymes.

### EXPERIMENTS

One aspect of the present invention is concerned with an improved system for producing glucoamylases and proteases. Small scale experiments were performed on petri dish to gather initial data. A larger scale experiment using a solid-state fermentation bioreactor was then performed.

### Inoculum Preparation

Aspergillus awamori and Aspergillus oryzae spores were inoculated on the surface of 1.7% (w/v) cornmeal agar in flasks. They were covered by sponge and placed in incubator for 5 days at 30° C. The spores were extracted by adding 10% glycerol solution and scraped with sterilized glass beans, followed by transferring the solution into the second and subsequent flasks so that the fungal solution could be extracted in a higher concentration. Spore count was later counted by using a Haemocytometer under a microscope.

### Bakery Wastes Preparation

Three bakery wastes including cake, bread and pastry were collected from Hong Kong Starbucks once a week. The bakery wastes were blended with the use of a blender, and kneaded into being homogeneous, and then were autoclaved. The bakery wastes were stored at −20° C. refrigerator for preservation.

### Initial Moisture Content

The sample of bakery was placed on pre-weighted aluminum foils which were subsequently weighted to 0.0001 g. The folded sample was then undergone freeze drying for overnight. After that, the freeze dried sample was weighted to 0.0001 g. The following equation was used for the calculation of the moisture content of the bakery waste, which was expressed as grams of water per gram of wet bakery waste.

$${M}_{db} = \frac{{W}_{i} - {W}_{f}}{{W}_{i}} \times 100$$

where,
M_{db}=Moisture content in wet basis (g water/g wet solid)
Wi=Total weight before drying (g)
W_{f}=Total weight after drying (g)
W_{d}=Weight of the dish (g)

### Fermentation Procedure

Petri dishes of 10 cm of diameter and 1 cm height were used to perform several preliminary result and micro scale effects of the experiment such as the effect of initial moisture content, addition of water and solvent of extraction during incubation. The petri dish experiment was acted as simpler system of solid state reactor. Autoclaved bakery wastes were packed at the petri dish evenly and then were inoculated with fungi, which was used as the culturing medium for the growth of fungi. Duplicate set of dishes per day (20 g bakery waste/dish) were inoculated with fungal spore with the concentration 5×10³ spores/g bakery waste and put into an incubator at 30° C. after cultivation.
Enzyme Extraction from Petri Dishes
Duplicate set of petri dishes will be selected to extract enzyme each day. The whole contents of fermented solid were blended with 4 mL Milli-q water. The mixture was placed into water bath for 30 minutes at 30° C. and swirled with a stirrer bar. The suspension was then centrifuged at 12,000 rpm for 10 minutes at 4° C. and the supernatant was collected by performing suction filtration. The filtrates were purified enzyme solutions and were stored at −200° C. refrigerator until used for enzymatic analysis.

### Enzymatic Activity Determination

### Glucoamylase Assay Analysis

The enzymatic extracts were diluted with 0.2 M sodium acetate buffer. Glucoamylase activity was assayed using gelatinized wheat flour 5% (w/v) as a substrate. Enzymatic reaction was performed by adding diluted enzyme solution to wheat flour suspension.
The mixture was incubated at 55° C. for 10 minutes. Trichloroacetic acid (TCA) (10% v/v) was used to quench the reaction at time 0 and 10 minutes. The resulting solutions were then centrifuged at 13,000 rpm for 10 minutes. The concentration of glucose in the collected supernatant was measured by using the Analox GL6 Glucose Analyzer and the enzyme activity is calculated by the following equation.
Unit definition: One enzyme activity unit (U) was defined as the amount of enzyme that releases 1 μmol of glucose per minute per the assayed conditions

$$Activity = \frac{{G}_{t ( 10 )} \times D - {G}_{t ( 0 )} \times D}{10}$$

where
Gₜ₍₁₀₎ is the glucose concentration of the mixtures after 10 minutes digestion.
Gₜ₍₀₎ is the glucose concentration of the mixtures before 10 minutes digestion
D is the dilution factor of the mixtures

### Protease Assay Analysis

The enzymatic extracts were diluted with water. Protease activity was assayed using either casein or wheat flour as substrate for enzymatic digestion of A. oryzae and A. awamori respectively. Enzymatic reaction was performed by adding diluted enzyme solution to casein or wheat flour suspension. The mixed reaction was incubated at room temperature for 30 minutes. Trichloroacetic acid (TCA) (10% v/v) was used to quench the reaction at time 0 and 30 minutes. Protease activity was determined by Free Amino Nitrogen (FAN) content measurement using the ninhydrin colorimetric method [30] and then absorbance measurement at 570 nm using spectrophotometer. Protease activity was calculated by the equation below.
One unit (U) of protease activity was defined as the amount of protease required to release 1 μg of FAN from substrate within one minute. (U=1 μg/min)

$$Activity = \frac{{FAN}_{t ( 30 )} \times D - {FAN}_{t ( 0 )} \times D}{30}$$

where FAN is the free amino nitrogen concentration of the reaction mixtures t(0) and t(30)
D is the dilution factor of the mixture

### Thermo Stability Test

The main purpose of the thermo stability test was to investigate the stability of enzyme activity at different temperatures. Enzyme activity can be adversely affected by temperature, through thermal deactivation which is crucial in the commercialization of the enzyme solution as a product. Therefore, kinetic studies on the multi-enzyme solutions are essential.
Irreversible thermal deactivation of glucoamylase was studied by incubating the enzyme solutions at particular temperatures in the absence of the substrate. Standard amounts of enzyme solution were placed into test tubes and located into water baths at temperatures between 30 and 70° C., with +5° C. increments for around 100 hours. Aliquots were withdrawn from the test tubes at different time intervals, cooled on ice for 1 hour, and then assayed at the normal enzyme assay conditions.

### Optimal Reaction Temperature and pH

The objective was to investigate the effects of different reaction temperature and pH on the enzyme activity of glucoamylase. A set of experiments were conducted to determine the optimal reaction temperature and pH of glucoamylase for A. awamori samples. The sample with the highest glucoamylase activity was chosen to conduct this experiment. For optimal pH determination, the enzyme extracts were diluted with 5 buffers with different pH values (from 3.5 to 7.5, with +1 increment) respectively. The diluted enzyme solutions were then undergone enzyme digestion at 55° C. using wheat flour as substrate. The glucoamylase activities were then determined by the glucose content measurement using the Analox GL6 Glucose Analyzer. For determination of optimal temperature, the pH of the diluted enzyme solutions were kept constant at 5.5 while reaction temperatures were varied from 40° C. to 75° C., with +5° C. increments and the enzyme activities were assayed with standard glucose assay.

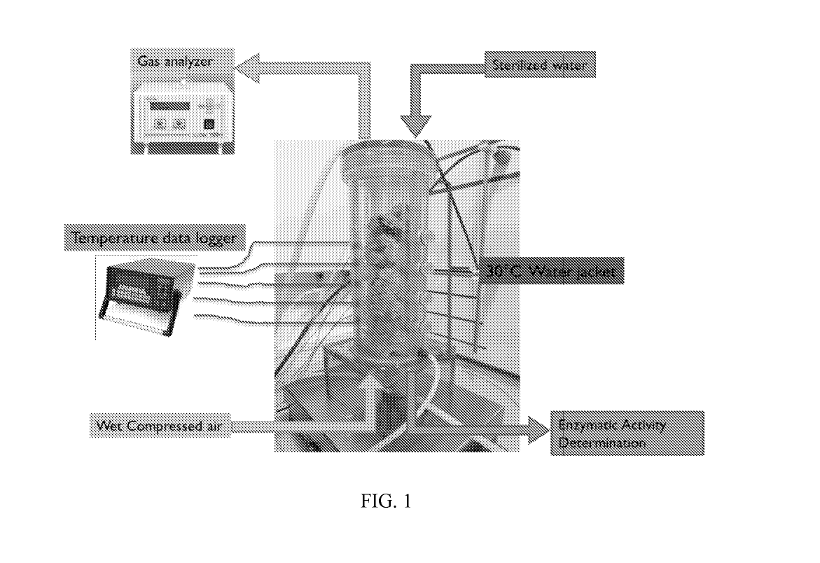

FIG. 1 shows an embodiment of a bioreactor of the present invention used in an experiment. The bioreactor is a packed bed solid state fermentation bioreactor. The bioreactor is cylindrical in profile defining a cavity for containing fermentation substrate. One characteristics of the packed bed solid state fermentation bioreactor is that it is provided with a moisture controlling means in the form of a spray nozzle. The spray nozzle is arranged at a top region of the bioreactor. The bioreactor is also equipped with means to supply air that passes packed bed of the fermentation substrate in the bioreactor. The bioreactor is provided with a gas analyzer for monitoring gas content during fermentation and hydrolysis of the fermentation substrate. The bioreactor is covered with a water jacket to maintain fermentation temperature at around 30° C. The fermentation temperature is being monitoring during operation. Filtered compressed and moistened air is first moistened and then introduced into the bioreactor from the bottom to supply oxygen and moisture as well as providing convective cooling. The spray nozzle is connected to a reservoir of autoclaved water through peristaltic pump to provide intermittent trickling of water to maintain moisture of substrates and to remove the heat produced by respiration of fungi. The gas analyzer and temperature data logger are connected to bioreactor to investigate and monitor the temperature change and O₂/CO₂ content throughout fermentation for monitoring the fungal growth.

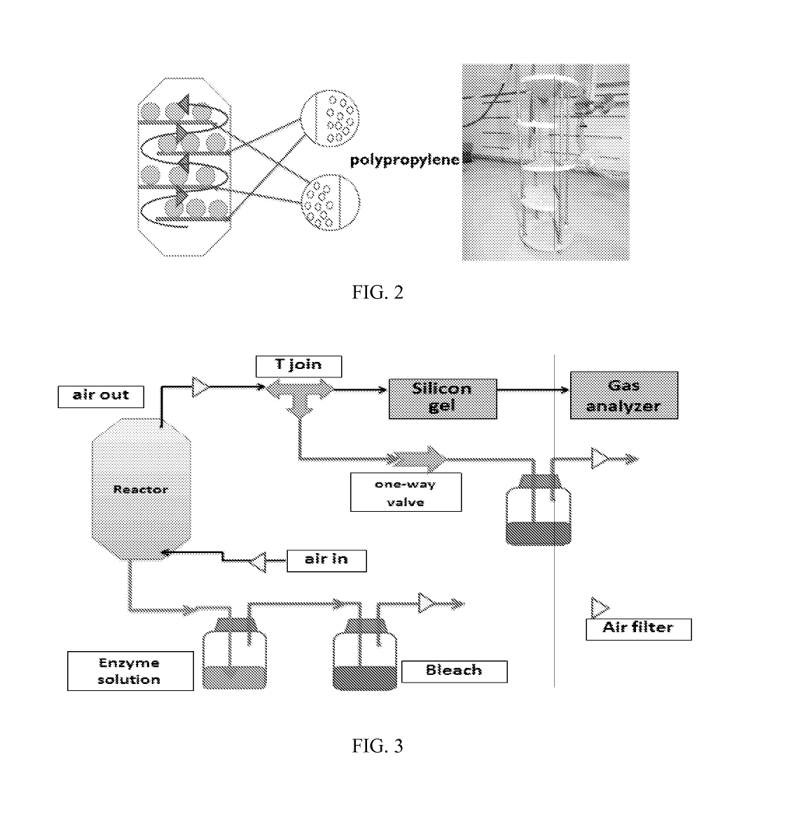

In a preferred embodiment of a bioreactor of the present invention, the bioreactor is provided with a number of shelves installed therein. The shelves take the form of laterally extending platforms for supporting the fermentation substrate. In a particular embodiment, the shelves are made of an inert material (e.g. polypropylene) with openings which allow water to pass. FIG. 2 shows a general construction of an embodiment of the shelves in the bioreactor. Studies show that this embodiment of fermentation bioreactor can enhance efficiency of fermentation and hence enzyme production.

In order to minimize contamination of the surrounding environment by fungi and fungal spores, a disinfected system was designed. FIG. 3 is a schematic diagram showing various mechanisms of the bioreactor including the disinfectant system.

As shown in FIG. 3, air exiting the system is filtered by air filter and sterilized by bleach before discharging to environment to prevent any contamination.

Aeration supplies oxygen to the microorganisms and removes carbon dioxide and the reparative heat of the medium by convective cooling while excessive aeration causes drying and thus decreases the moisture content of the medium which is not favorable for fungal growth.

Moisture content of the substrates plays a significant role for fungal growth as it provides stability for compounds and affects diffusion of bio-chemicals. Moisture level fluctuates during fermentation as the result of consumption or metabolic water production of microorganism, evaporation due to metabolic heat and evaporative cooling. Therefore, intermittent trickling of water is required to compensate the moisture loss to maintain the optimal moisture content.

Particle size and surface area have effect on aeration rate, mass transfer and fungal growth. In large particles, the decreased surface area reduces liquid holding capacity and aeration rate, and thus has negative effect on production formation.

In an experiment, selected bakery waste was first mixed by blender and autoclaved for sterilization. Moisture conditioning was performed on the bakery waste to maintain initial moisture content of 65-70%. It then was rolled into particles with predetermined particle size and was inoculated with the fungal spore solution (1×10⁵ spores/g dry solid). Inoculated substrates were placed on the shelf evenly and then put on the shelves of the packed bed bioreactor for fermentation.

At different time intervals, around 3-5 g of samples was taken out from sampling ports at different height of the bioreactor. The samples were first mixed with mili-q water with the ratio of 20 ml water/g solid. The mixture was placed into water bath for 30 minutes at 30° C. and swirled with a stirrer bar to extract the enzyme from the samples. The suspension was then centrifuged at 12,000 rpm for 10 minutes at 4° C. and suction filtration was performed to collect the supernatant. The filtrates were purified enzyme solutions and were stored at −20° C. refrigerator for preservation until enzyme activity analysis is conducted.

Data collected form Glucoamylase and Protease essay was used to plot the graph showing enzyme activity trend for further analysis.

Aeration rate was optimized for glucoamylase production. As cake waste gives the highest glucoamylase activity, cake was selected as the substrate. The cake was rolled into particles with the diameter of 1-1.5 cm and the moisture content was conditioned to 65-70% dry weight. Then the substrate was inoculated with A. awomori spores with the concentration of (1×10⁵ spores/g dry cake). Two experiments with aeration rate of 1.6 vvm and 1 vvm was carried out to investigate the effect of aeration rate change on glucoamylase activity.

Experiments were conducted to determine whether or which food waste would be best as fermentation substrate.

### Glucoamylase Activity

Experiments were conducted to ascertain whether cake waste, bread waste and pastry waste are effective fermentation substrate to culture Aspergillus awamori, and for the production of glucoamylase. The experiments were conducted in petri-dish environment.

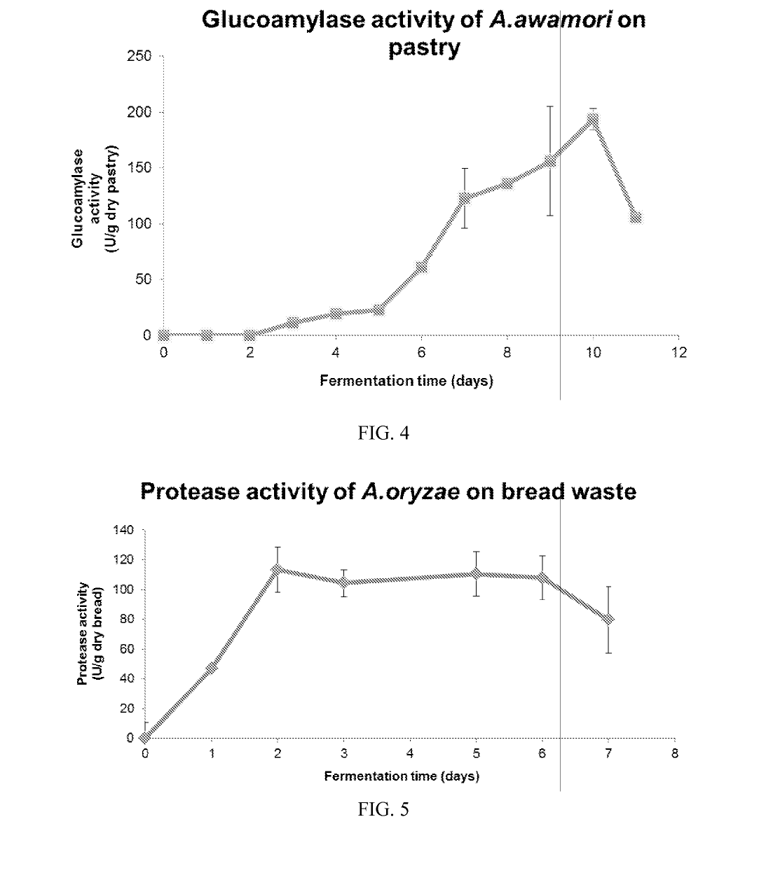

FIG. 4 shows glucoamylase activity of A. awamori cultivated on pastry waste during different phases in development. FIG. 4 shows the glucoamylase activity of A. awamori culturing on pastry waste against the fermentation time.

Similar experiments were conducted by using waste cake and bread as culture medium. The maximum enzyme activity and their corresponding productivity of each substrate were calculated. The data is summarized in the following table.

TABLE 4
Maximum glucoamylase activity and productivity of enzyme
produced by A. awamori on each substrate

| | Cake | Bread | Pastry |
|---|---|---|---|
| Maximum | 229.6 ± 3.6 | 205.1 ± 10 | 193.5 ± 9.6 |
| glucoamylase activity | | | |
| (U/g dry bakery | | | |
| waste) | | | |
| Productivity (U/g dry | 25.5 ± 0.4 | 29.3 ± 1.4 | 19.4 ± 0.96 |
| bakery waste-day) | | | |

From the above table, it is shown that while all bakery wastes are suitable candidates as fermentation substrate, cake waste shows the most outstanding result in that it can produce the highest glucoamylase activity and productivity. It shows that cake waste is the most suitable substrate among the three bakery wastes to support glucoamylase production of A. awamori.

### Protease Activity

Similar experiments were conducted to determine the use of bakery waste to culture Aspergillus oryzae.
The protease activities of A. oryzae cultivated on bread is shown in FIG. 5. Different from glucoamylase, protease activity increased almost immediately after inoculation. The absence of a lag phase indicates that the fermentation conditions used were conductive to growth. A high protease activity was achieved only after 3 days of inoculation for bread waste and the high activity at around 220 U/g bread was maintained until day 6.

Similar experiments were conducted by using waste cake and bread as culture medium. The maximum enzyme activity and their corresponding productivity of each substrate were calculated. The experimental results are summarized in the following table.

TABLE 5
Maximum protease activity and productivity of enzyme
produced by A. oryzae on each substrate

| | Cake | Bread | Pastry |
|---|---|---|---|
| Maximum protease | 138.1 ± 12.1 | 113.2 ± 15.1 | 147.2 ± 18.8 |
| activity (U/g dry | | | |
| bakery waste) | | | |
| Productivity (U/g dry | 13.8 ± 0.24 | 56.5 ± 7.6 | 21.0 ± 2.68 |
| bakery waste-day) | | | |

It is shown that pastry waste shows the most outstanding result for protease production. The maximum protease activity was at 147.2 U/g dry pastry. It shows that pastry waste is the most suitable substrate among the three bakery wastes to support protease production of A. oryzae.

Experiments were conducted to demonstrate the effect of pH and temperature on glucoamylase.

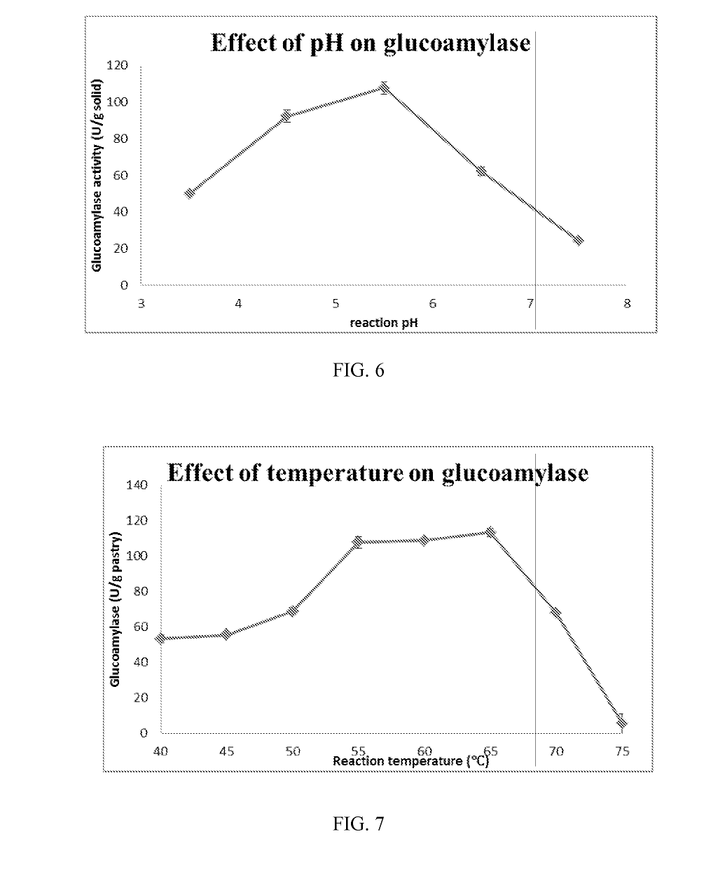

FIG. 6 shows the relationship between glucoamylase activity and pH during fermentation of pastry waste. The glucoamylase activity increased gradually from pH 3.5 and reached a maximum value at pH 5.5. Then the glucoamylase activity decreased significantly. It is shown that maintaining the pH in the range of 4.5-6 can increase production of the enzyme.

FIG. 7 shows the relationship between glucoamylase activity and reaction temperature. The glucoamylase activity increased from 40° C. to 55° C. Then it remained fair constant at 110 U/g pastry and reached a maximum value at 65° C. Then, there was a rapid decrease of glucoamylase activity beyond 65° C. The activity dropped to almost zero at 75° C. It is shown that maintaining the operating temperature in the range of 50-70° C., or preferably 55-65° C. can increase production of the enzyme.

Further experiments were conducted to determine thermo-stability of glucoamylase at temperatures 55, 60 and 65 by calculating the half-lives. A longer half-life indicates higher thermo-stability.

TABLE 6
Half-lives of glucoamylase at temperatures 55° C., 60° C. and 65° C.

| Temperature (° C.) | Half-life, t_{1/2} (hours) |
|---|---|
| 55 | 49.51 |
| 60 | 0.38 |
| 65 | 0.32 |

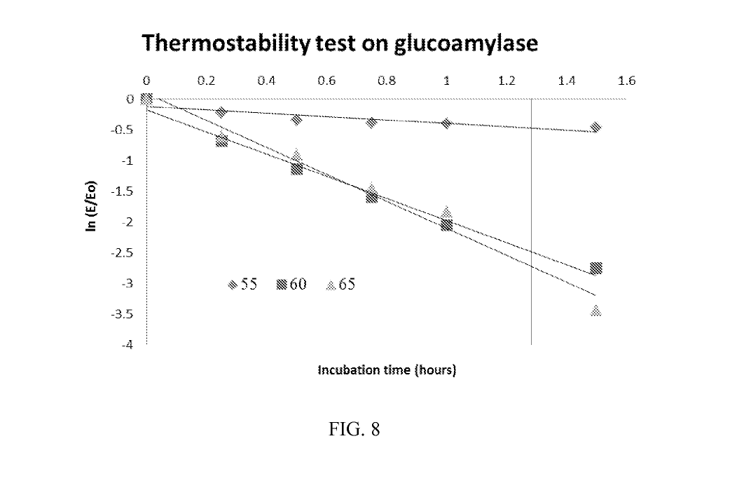

FIG. 8 shows that glucoamylase has the highest thermo-stability at 55° C. as the half-life is the longest. It shows that the optimal reaction temperature and pH for the enzymatic hydrolysis of food waste by glucoamylase is 55° C. and 5.5, respectively.

Experiments were conducted to determine the level of glucose released during fermentation of bakery waste by A. awamori. Release of the glucose indicates that fermentation is proceeding properly.

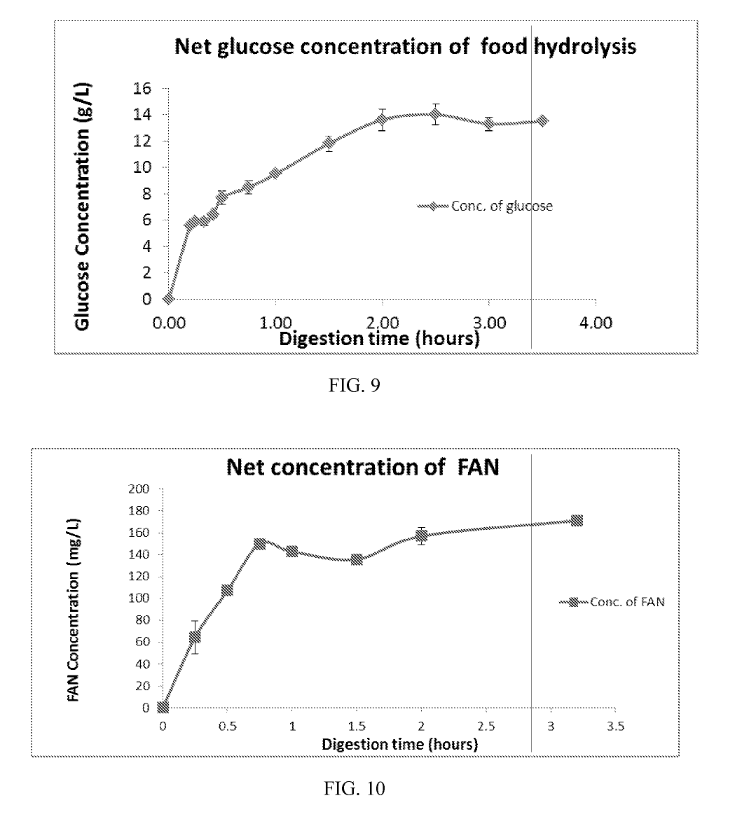

FIG. 9 shows during fermentation of bakery waste by A. awamori the production of glucose over time.

Experiments were conducted to determine the production of amino acid by the enzyme of A. oryzae over time. This was achieved by measuring the nitrogen content of free amino nitrogen (FAN). FIG. 10 shows the amount of FAN produced over time during fermentation of bakery waste.

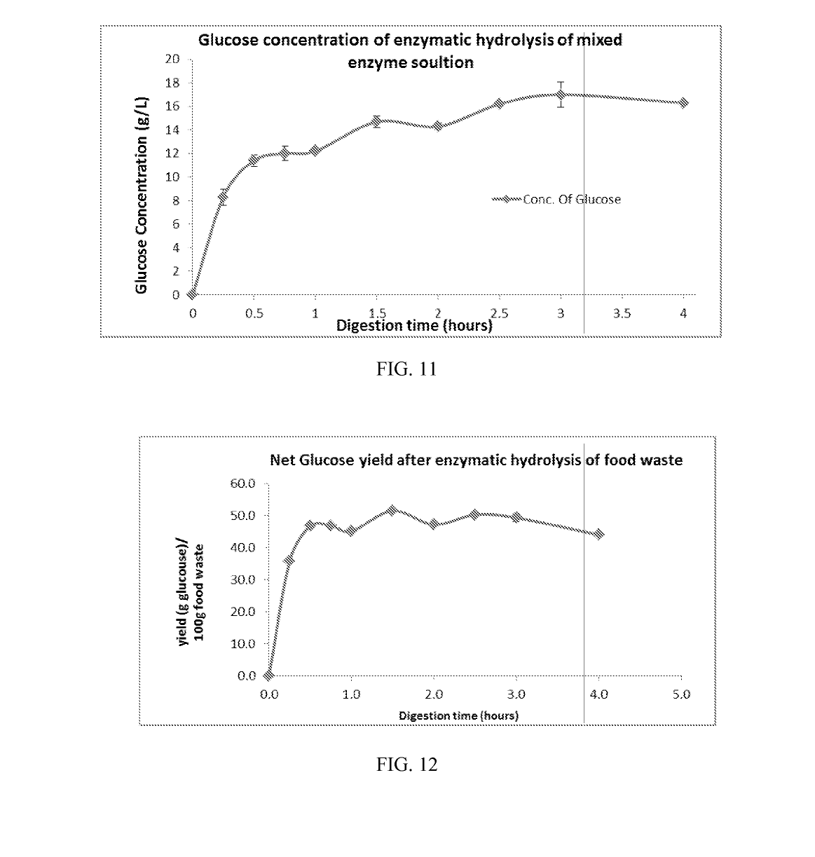

Experiments were conducted to determine the production of glucose by enzyme from A. awamori and A. oryzae during fermentation of bakery waste. FIG. 11 shows the concentration of glucose over time during the fermentation. It is shown that the glucose concentration reached the highest level at 18.0 g/L.

FIG. 12 shows that the maximum net amount of glucose obtained was 51.5 g per 100 g food waste being hydrolyzed by using enzyme from A. awamori and A. oryzae.

Experiments were conducted to determine the difference between using only A. awamori and using A. awamori and A. oryzae for fermentation.

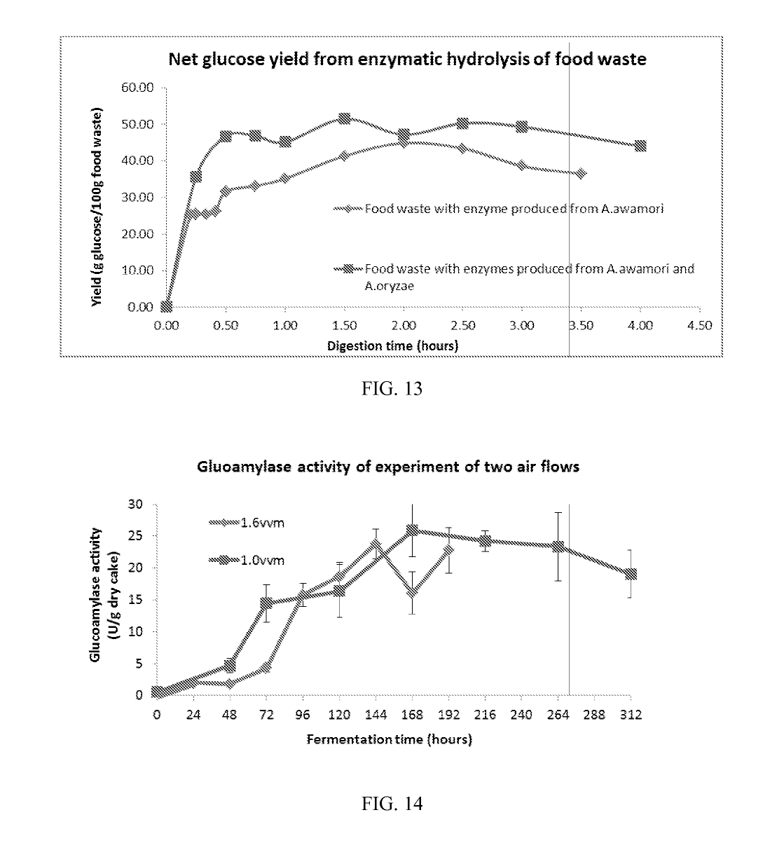

FIG. 13 shows that the net glucose yield of food hydrolysis using enzymes produced from both fungi was higher than the one using the enzymes produced from A. awamori only. This suggests the concurrent use of both these enzymes during fermentation of food waste.

The following table summarizes the rates of conversion of protein to FAN and carbohydrate to glucose when using one of A. awamori and A. oryzae, and both of A. awamori and A. oryzae.

TABLE 7
The conversion rate of each enzymatic hydrolysis experiment

| Food hydrolysis using | Conversion | Conversion |
|---|---|---|
| enzyme produced | rate of | rate of carbohydrate |
| from: | protein to FAN | to glucose |
| A. awamori | — | 74.12% |
| A. oryzae | 6.15% | — |
| A. awamori and | 38.45% | 95.30% |
| A. oryzae | | |

These results shows that simultaneously use of A. awamori and A. oryzae can unexpectedly enhance the fermentation rate and production of the enzymes, namely glucoamylase and protease.

Experiments were conducted to determine the effect of air flow and production of the enzymes.

FIG. 14 shows the effects of two different air flows on glucoamylase activity. When the air flow was at 1.6 vvm, the activity of glucoamylase continued to increase during the first 8 days of the experiment. Similar glucoamylase activity was observed when the air flow was at 1 vvm.

From the above illustration, it is demonstrated that the present invention concerning the design of a packed bed fermentation bioreactor system can effectively produce glucomylases and/or proteases.

It should be understood that certain features of the invention, which are, for clarity, described in the content of separate embodiments, may be provided in combination in a single embodiment. Conversely, various features of the invention which are, for brevity, described in the content of a single embodiment, may be provided separately or in any appropriate sub-combinations. It is to be noted that certain features of the embodiments are illustrated by way of non-limiting examples. Also, a skilled person in the art will be aware of the prior art which is not explained in the above for brevity purpose.

1. A system of production of glucoamylases and/or proteases, comprising a packed bed solid state fermentation bioreactor, wherein said bioreactor is adapted to contain and to operate using organic waste as fermentation substrate, and is provided with:
spray nozzle for controlling moisture content of the fermentation substrate;
means for supplying air to pass through packed bed of the fermentation substrate in said bioreactor;
means for analyzing gas content in said bioreactor;
means for monitoring temperature in said bioreactor;
means for controlling temperature of said bioreactor; and
a port via which a sample in said bioreactor is obtainable; and
means for harvesting enzymatic solution from said bioreactor, the enzymatic solution containing the glucoamylases and/or proteases.

2. A system as claimed in claim 1, wherein said bioreactor is adapted to receive and operate using at least one of Aspergillus awamori and Aspergillus oryzae as fermentation agent.

3. A system as claimed in claim 1, wherein the fermentation substrate is bakery waste.

4. A system as claimed in claim 3, wherein the bakery waste includes at least one of bread waste, cake waste and pastry waste.

5. A system as claimed in claim 1, wherein the spray nozzle is adapted to introduce sterilized water in said bioreactor.

6. A system as claimed in claim 5, wherein the spray nozzle is arranged at top region of said bioreactor.

7. A system as claimed in claim 5, wherein the spray nozzle is adapted to maintain moisture content of the fermentation substrate in said bioreactor at 60-80 wt %.

8. A system as claimed in claim 1, wherein said air supply means includes filtering means to prevent contamination.

9. A system as claimed in claim 8 wherein said air supply means is adapted to introduce filtered air from bottom region of said bioreactor.

10. A system as claimed in claim 1, comprising means for receiving air exiting said bioreactor.

11. A system as claimed in claim 9, wherein said air receiving means is connected to, and is upstream of said air analyzing means.

12. A system as claimed in claim 11, comprising an outlet at a top region of said bioreactor via which air exits to said air receiving means.

13. A system as claimed in claim 1, wherein said air supply means is adapted to moist air before introducing air in said bioreactor.

14. A system as claimed in claim 1, wherein said temperature control means is in the form of a water jacket for removing heat generated during operation of said system.

15. A system as claimed in claim 1, wherein said bioreactor is provided with a port via which the enzymatic solution exits.

16. A system as claimed in claim 15, wherein said port is arranged at a bottom region of said bioreactor.

17. A system as claimed in claim 1, wherein the proteases includes at least one of serine proteases, cysteine proteases, aspartic proteases and metalloproteases.

18. A system as claimed in claim 1, wherein said fermentation reactor is of cylindrical profile defining a cavity to contain the fermentation substrate.

19. A system as claimed in claim 18, wherein the fermentation substrate is provided with a plurality of platform members laterally extending from side wall of said fermentation reactor and spaced apart from each other, said platform members for supporting the fermentation substrate.

20. A method of producing glucoamylases and/or proteases, comprising steps of:
providing a packed bed solid state fermentation bioreactor defining a cylindrical body;
providing organic waste as fermentation substrate for use in the bioreactor;
providing platform members in the bioreactor and laterally extending from side wall of the bioreactor, the platform members supporting the fermentation substrate and spaced apart from each other;
introducing Aspergillus awamori and Aspergillus oryzae in the bioreactor for producing enzymes acting as biocatalysts for solid state fermentation using the organic waste;
providing a spray nozzle for controlling moisture content of the fermentation substrate;
supplying filtered air to pass through packed bed of the fermentation substrate in said bioreactor;
analyzing gas content in the fermentation bioreactor;
monitoring temperature in fermentation bioreactor;
controlling temperature of the fermentation bioreactor;
obtaining sample from via a port via of the fermentation bioreactor for monitoring purpose;
allowing fermentation to take place; and
harvesting enzymatic solution from the bioreactor, the enzymatic solution containing the glucoamylases and/or proteases.
